(19) United States
(12) Patent Application Publication
(10) Pub. No.: US 2008/0262483 A1
(43) Pub. Date: Oct. 23, 2008

(54) METHOD FOR REMOVING PERMANENT TISSUE MARKINGS

(75) Inventors: Christopher C. Capelli, Kenosha, WI (US); Michael B. Chancellor, Pittsburgh, PA (US)

Correspondence Address: ECKERT SEAMANS CHERIN & MELLOTT, 600 GRANT STREET, 44TH FLOOR, PITTSBURGH, PA, 15219, US

(73) Assignee: University of Pittsburgh-Of the Commonwealth System of Higher Education, Pittsburgh, PA (US)

(21) Appl. No.: 12/103,342

(22) Filed: Apr. 15, 2008

Related U.S. Application Data
(60) Provisional application No. 60/923,897, filed on Apr. 17, 2007.

(51) Int. Cl. A61B 17/50; A61N 7/00; A61B 18/20

(52) U.S. Cl. 606/9; 606/131; 601/4

(57) ABSTRACT

A method of removing markings, such as a tattoos, from the tissue of a subject that includes steps of generating one or more shock wave pulses external to the subject's body and focusing the one or more shock wave pulses within one or more portions of the tissue, each of the one or more portions of the tissue containing at least a portion of the markings. The markings are typically caused by a plurality of pigment particles contained in the tissue, and the focusing step causes fragmentation of the particles so that they can be eliminated by the body.

### CROSS-REFERENCE TO RELATED APPLICATIONS

This application claims the benefit of U.S. Provisional Application No. 60/923,897, entitled “Lithotripsy for Removal of tissue Markings,” which was filed on Apr. 17, 2007, the disclosure of which is incorporated herein by reference.

### FIELD OF THE INVENTION

The present invention relates to the removal of concretions, such as tattoos, located within the dermis of a subject, and in particular to the removal of such concretions using shock wave pulses generated external to the subject's body.

### BACKGROUND OF THE INVENTION

Humans have been applying tattoos to the skin for over 8000 years. The inks and dyes used were historically derived from substances found in nature and typically comprise a heterogeneous suspension of pigmented particles and other impurities. A well-known example is India ink, which is a suspension of carbon particles in a liquid.

Tattoos are produced by applying tattoo ink into the dermis, where the ink remains permanently. This technique introduces the pigment suspension through the skin by an alternating pressure-suction action caused by the elasticity of the skin in combination with the up-and-down movement of tattoo needles. Water and other carriers for the pigment introduced into the skin diffuse through the tissues and are absorbed. The insoluble pigment particles are deposited in the dermis generally where initially placed. After being tattooed into the skin, the pigment particles and agglomerates thereof are exclusively found intracytoplasmatically, lying in the membrane-bound structures of the dermis cells known as secondary lysosomes. This occurs due to active phagocytosis of the pigment into the dermal cells (i.e., by macrophages and/or fibroblasts). The resulting pigment agglomerates range up to a few micrometers in diameter.

Once the skin has healed, most pigment particles remain in the interstitial space of the tissue. Inks used for tattooing resist elimination by virtue of their inertness and the relatively large size of the insoluble pigment particles. A tattoo produced in this manner may partially fade over time but will generally remain present throughout the life of the tattooed person.

Studies have been performed analyzing the make-up of tattoo inks. In one study, samples of 30 tattoo inks were examined using “standardless” energy-dispersive spectrometry. Of the 30 tattoo inks studied, the most commonly identified elements were aluminum (87% of the pigments), oxygen (73% of the pigments), titanium (67% of the pigments), and carbon (67% of the pigments). In addition, the relative contributions of elements to the tattoo ink compositions were highly variable between different compounds.

In another study, it was found that the diameters of the pigments vary from about 20 nm to 900 nm. Transmission electron microscopy pictures of the pigments showed a variety of shapes such as needles, platelets, cubes, bars, and a number of irregular shapes. Besides primary particles, aggregates composed of primary particles grown together at their surfaces and agglomerates (groups of single crystals joined together at their edges) were present in the same picture.

In all types of conventional tattooing (decorative, cosmetic, and reconstructive), once the pigment has been administered into the dermis to form a tattoo, the pigment generally remains permanently in place. However, many people have a change of heart after being tattooed. For example, a person may desire to remove or change the design of a decorative tattoo. Alternatively, an individual with cosmetic tattooing, such as eyeliners, eyebrows, or lip coloring, may wish to change the color or area tattooed as fashion changes.

Unfortunately, there is currently no simple and successful way to remove tattoos. One approach that has been proposed (described in United States patent Application Publication Number 2003/0167964) is the use of tattoo inks that are removable on demand. These inks consist of microparticles that are constructed with specific electromagnetic absorption and/or structural properties that facilitate changing and/or removal by applying specific energy (such as electromagnetic radiation from a laser or flash-lamp). In other embodiments, the pigment vehicles composed of the pigment are such that they are susceptible to a specific externally applied energy source, such as thermal, sonic (ultrasound), light (e.g., laser light, infrared light, or ultraviolet light), electric, magnetic, chemical, enzymatic, mechanical, or any other type of energy or combination of energies. The problem with this approach is that it requires tattoos, or skin markings, to use these new types of ink. Such inks, however, have not been widely used to date. In addition, tattoos using conventional inks, which are the majority of tattoos, still present the difficulties in removal as discussed above.

Current approaches to the removal of traditional tattoos include salabrasion, cryosurgery, surgical excision, and CO₂-laser treatment. However, these methods require invasive procedures associated with potential complications, such as infections, and often result in conspicuous scarring.

More recently, the use of Q-switched lasers has gained wide acceptance and has revolutionized the treatment of tattoos. By restricting pulse duration, ink particles reach very high temperatures with relative sparing of adjacent normal skin. This significantly decreases the scarring that often results after nonselective tattoo removal methods, such as dermabrasion or treatment with CO₂-lasers. The mechanisms for tattoo removal by Q-switched laser radiation are poorly understood. It is thought that the use of Q-switched laser radiation allows for more specific removal of tattoos by the mechanisms of selective photothermolysis and thermokinetic selectivity.

While, as discussed above, the Q-switched laser has been revolutionary in the removal of tattoos, it is far from perfect. In general, laser treatment, including treatment with a Q-switched laser, is painful. Use of a local injection with lidocaine or topical anesthesia cream typically is used prior to laser treatment. Additionally, common adverse effects following laser tattoo treatment with Q-switched lasers include textural change, scarring, pigmentary alteration, and transient hypopigmentation. The development of localized and general allergic reactions following tattoo removal with the Q-switched laser may, although rare, also be possible.

In addition, some tattoos are clinically resistant to all laser therapies despite the predicted high particle temperatures achieved through selective photothermolysis. Reasons cited for failure of some tattoos to clear include the absorption spectrum of the pigment, the depth of pigment, and the structural properties of the ink. Also, some inks may remain in the dermis after being rephagocytosed by resident cells.

Finally, laser removal requires multiple treatment sessions (usually five to twenty) with expensive equipment for maximal elimination. Typically, since many wavelengths are needed to treat multicolored tattoos, no single laser system can be used alone to remove all the available inks and combinations of inks. As a result, the overall cost of laser removal is generally prohibitively expensive. Even with multiple treatments, laser therapy is usually limited to eliminating only from 50-70% of the tattoo pigment, resulting in a residual smudge.

There is thus a need for a non-surgical method for removal of tissue markings that is not dependent on the transmission and absorption of laser light.

### SUMMARY OF THE INVENTION

The invention provides, in one embodiment, a method of removing markings, such as a tattoos, from the tissue of a subject that includes steps of generating one or more shock wave pulses external to the subject's body and focusing the one or more shock wave pulses within one or more portions of the tissue, each of the one or more portions of the tissue containing at least a portion of the markings. The markings are typically caused by a plurality of pigment particles contained in the tissue, and the focusing step causes fragmentation of the particles so that they can be eliminated by the body.

In one particular embodiment, the one or more shock wave pulses have a delivered positive pulse pressure wave of greater than 1.5 MPA and a pulse duration of less than 5 μs. In another particular embodiment, the one or more shock wave pulses have a delivered positive pulse pressure wave of greater than 10 MPA and a pulse duration of less than 1 μs. In other particular embodiments, the one or more shock wave pulses have a delivered negative pulse pressure wave of less than 20 MPA and less than 10 MPA, respectively.

In another embodiment, a location of the focusing of each of the shock wave pulses is changed so that no consecutive shock wave pulses are focused within the same portion of the tissue.

The generating step may be performed using any suitable transducer, such as, without limitation, an ultrasonic transducer, an opto-acoustic transducer, or a spark-gap transducer.

In another embodiment, the invention provides a method of removing markings from the tissue of a subject including steps of selecting an initial target location of the tissue containing a first portion of the markings, generating a first shock wave pulse external to the subject's body, focusing the first shock wave pulse within the tissue at the initial target location, selecting a new target location of the tissue containing a second portion of the markings, the new target location being different from the initial target location, generating a second shock wave pulse external to the subject's body, and focusing the second shock wave pulse within the tissue at the new target location.

Still another embodiment of the invention provides a method of comminuting a concretion located within the dermis of a subject including steps of generating one or more shock wave pulses external to the subject's body and focusing the one or more shock wave pulses within one or more portions of the dermis where the concretion is located.

Therefore, it should now be apparent that the invention substantially achieves all the above aspects and advantages. Additional aspects and advantages of the invention will be set forth in the description that follows, and in part will be obvious from the description, or may be learned by practice of the invention. Moreover, the aspects and advantages of the invention may be realized and obtained by means of the instrumentalities and combinations particularly pointed out in the appended claims.

### BRIEF DESCRIPTION OF THE DRAWINGS

The accompanying drawings illustrate presently preferred embodiments of the invention, and together with the general description given above and the detailed description given below, serve to explain the principles of the invention. As shown throughout the drawings, like reference numerals designate like or corresponding parts.

FIG. 1 is a block diagram of an apparatus for removing tissue markings, such as tattoos, and other concretions located within the dermis of a subject according to one non-limiting embodiment of the invention;

FIGS. 2 and 3 are flowcharts of methods for removing tissue markings, such as tattoos, and other concretions located within the dermis of a subject according, according to two particular embodiments of the present invention.

### DESCRIPTION OF THE PREFERRED EMBODIMENTS

Traditionally, extracorporeal shockwave lithotripsy, or “lithotripsy,” has been used in the removal of kidney stones. Lithotripsy uses shock waves to pulverize urinary calculi (kidney stones) non-invasively. As used herein, a shock wave shall refer to a compressional wave of high amplitude caused by a shock to the medium (such as air, water, or a solid) through which the wave travels. A shock wave is characterized by a very rapid, sudden pressure increase in the transmission medium and is quite different from ultrasound. In lithotripsy, the shock waves are transmitted through the patient's skin and pass harmlessly through the patient's soft tissue. The shock waves then pass through the kidney and strike the stone. At the stone boundary, energy is lost, and this causes small cracks to form on the edge of the stone. The same effect occurs when the shock waves exit the stone. With successive shocks, the cracks open up, and in turn, smaller cracks form within the large cracks. Eventually, the stone is reduced to small particles, which are then flushed out of the kidneys or ureter naturally during urination.

The present invention provides of a method for the removal of permanent tissue markings, such as tattoos and other concretions located within the dermis of a subject, through the use of extracorporeally generated shock waves which are employed to efficiently destroy (comminute) the particles which form the concretion, such as the pigment particles which form tattoos.

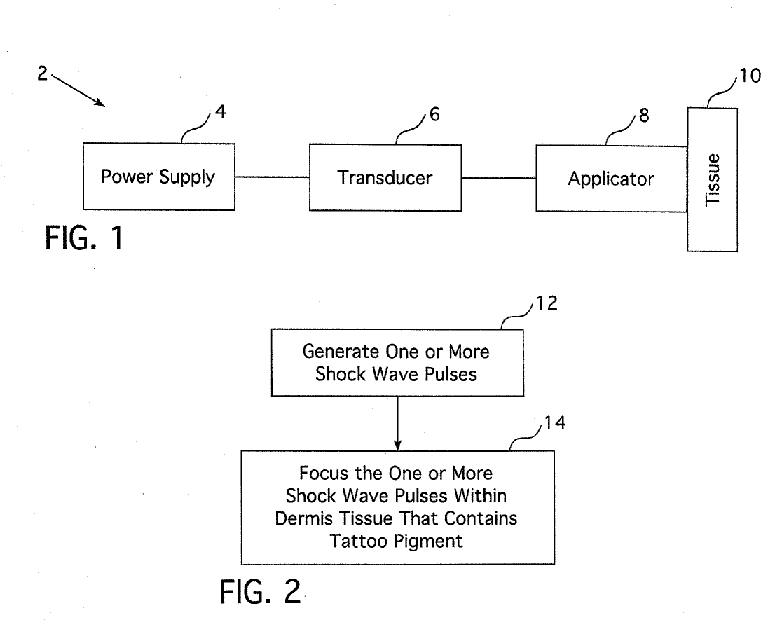

FIG. 1 is a block diagram of an apparatus 2 for removing tissue markings, such as tattoos, and other concretions located within the dermis of a subject according to one non-limiting embodiment of the invention. The apparatus 2 includes a power supply (generator) 4, a transducer 6, and an applicator 8 coupled to the transducer 6. The power supply (generator) 4 converts energy such as a standard household current to high frequency electrical energy. This high frequency electrical energy is transmitted to the transducer 6 which uses the high frequency electrical energy to produce the shock wave pulses. The applicator 8, which may include a plastic housing for holding the transducer 6, couples the transducer 6 with the tissue 10 of the subject so as to have an efficient transmission of the shock wave pulses to the specific site being treated (i.e. the site with the tissue marking or other concretion). As described in more detail herein, the shock wave pulses are focused in the tissue 10 so as to cause the removal of the tissue marking or other concretion.

As discussed above, the transducer 6 generates the shock waves that are employed in the present invention. Currently, there are at least three different types of known shock wave transducers that may be employed as the transducer 6 and include the spark gap transducer, the piezoelectric transducer and the opto-acoustic transducer. Descriptions of each of these types of transducers are provided below. The listed transducer types are not, however meant to be limiting, and it is to be understood that any transducer that is able to develop an adequate shock wave that can fragment a concretion in the dermis may be used in the present invention.

Spark gap transducers are currently employed in shock wave lithotripsy for the treatment of kidney stones. U.S. Pat. No. 5,195,508 provides a general description of spark gap transducers. Spark gap transducers consist of electrodes that develop a spark underwater to produce sound energy. Utilizing electro-hydraulic principles, water at the tip of the electrode is vaporized by an electrical discharge up to 30,000 volts which creates a pressure wave that bounces off an ellipsoid, reconverging at an outside point. While suitable for use in the present invention, spark gap transducers have the disadvantage that the shock waves generated by spark gaps are reproducible only with difficulty and, consequently, may be metered also with difficulty. Further, spark gap transducers suffer from having a very short service life, thus needing replacement often.

Piezoelectric transducers are also currently employed in shock wave lithotripsy for the treatment of kidney stones. Typical of such transducers are cup-shaped or planar transducers as described in U.S. Pat. Nos. 4,526,168; 4,858,597; 5,193,527; and 5,015,929. In other such transducers, the ultrasonic shock waves are focused by electronic or acoustic means. Piezoelectric transducers have the advantage that the pulses which they generate may be reproduced and metered perfectly and that their service life, subject to appropriate construction, is considerably greater than that of spark gap transducers.

For the present invention, shock waves may also be generated using ultrasonic transducers in the form of spheroidal caps. The main object of spheroidal caps is to concentrate the sonic energy emitted by a piezoelectric transducer on a minimum cross-section and in limiting the required total output. Piezoelectric transducers in the form of spheroidal caps are commonly produced as piezoceramic appliances, e.g. based on barium titanate, by being pressed into shape, sintered and then polarized radially.

It should be noted that there are numerous medical devices that use ultrasound waves generated by piezoelectric transducers. The key difference is that the ultrasound devices currently available for non-lithotripsy purposes are designed to provide continuous pulses of lower energy. The pulse energies required to produce sufficient energy to fragment particles using such devices would be high. As a result, the application of continuous ultrasonic oscillation from standard ultrasonic devices to a concretion formed within the body is impossible because burning of normal healthy body tissue in the vicinity of the concretion would be unavoidable at the high energy density required.

Opto-acoustic transducers convert pulsed optical energy to acoustic energy in order to generate shock waves. Opto-acoustic transducers are described in, for example, U.S. Pat. No. 5,071,422; 6,379,325; 6,491,685.

U.S. Pat. No. 6,379,325 discloses how a particular opto-acoustic transducer, which converts pulsed optical energy to acoustic energy in a liquid ambient medium, functions when used in ultrasound thrombolysis and angioplasty. The pulsed optical energy is most conveniently provided by a laser source operating at the desired repetition rate. The laser wavelength can be chosen to operate anywhere from 200 nm to 5000 nm such that the wavelength can be transmitted efficiently through a fiber optic. Pulse energy density may vary from 0.01 J/cm² to 4 J/cm², pulse duration from 3 ns to 1 us, and pulse frequency, or repetition rate may vary from 10 Hz to 100 kHz.

U.S. Pat. No. 5,071,422, describes how opto-acoustic transducers function when used in ultrasound lithotripsy. This patent discloses that, for optimal use, the pulses are at wavelengths corresponding to wavelengths for which the object has a relatively shallow depth of penetration. Preferably, wavelengths between 350 and 550 nanometers are used for urinary calculi (most preferably 251, 504, or 450 nanometers). The laser is preferably of the pulsed dye type for relatively long pulse durations but may be of other types. The pulses have durations of at least 10 nanoseconds (preferably between 0.05 and 2 microseconds), and the pulse energy is no greater than 0.200 joules, preferably between 0.005 and 0.200 joules. The fiber is flexible and has a core diameter no greater than 1000 microns, preferably between 60 and 600 microns, and more specifically 200 microns. The distal end of the fiber is in contact with the object (a stone) and the interface between them is surrounded by fluid. The laser pulses are applied in brief bursts, preferably greater than 10 hertz, and remaining fragments are broken down by one-shot pulses. The duration of each pulse delivered by the laser is chosen to minimize the energy delivered to the stone while still accomplishing fragmentation (i.e., the breaking down of the stone into smaller particles). The threshold energy level decreases with decreasing pulse duration. Because lower energy pulses are less likely to cause thermal damage or to propel the stone or the broken off particles into surrounding tissue, pulse durations of less than 10 microseconds, preferably between 0.05 and 2.0 microseconds and preferably between 0.5 and 2.0 microseconds.

Based on what has been discussed in the prior art, the preferred embodiment of the present invention employs an opto-acoustic transducer as the transducer 6.

The present invention provides a method of removing concretions located within the dermis of a subject, such as, without limitation, tissue markings caused by pigments that are placed in dermis tissue, that employs shock wave pulses that are focused within the dermal layer that contains the concretion, e.g., the pigment. Preferably, the method employs an apparatus such as apparatus 2 described above, to produce the shock wave pulses. The shock waves are transmitted into the patient's skin and pass harmlessly through the patient's soft tissue. The shock wave pulses then cause the destruction of the concretion (e.g., the pigment particles) within the dermis.

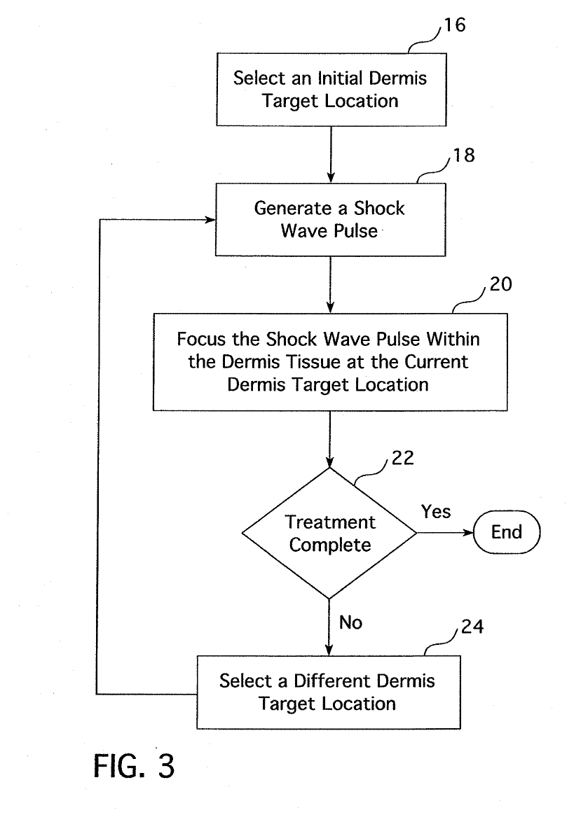

One particular method according to one embodiment of the present invention is shown in FIG. 2. For illustrative purposes, the method is described in connection with the removal of a tattoo. It should be understood, however, that this is not meant to be limiting, and that the method may be employed to remove other concretions located within the dermis of a subject. In addition, the method is also described in connection with the apparatus 2 shown in FIG. 1. Again, this is not meant to be limiting and it is to be understood that the method may be practiced with other suitable apparatus capable of generating and focusing suitable shock waves.

The method begins at step 12, wherein one or more shock wave pulses are generated extracorporeally using the apparatus 2. Then, in step 14, those shock wave pulses are focused within the dermis tissue of the subject that contains the tattoo pigment, preferably by applying the applicator 8 to the surface of the subject's skin in the appropriate places. The focusing of the shock waves causes fragmentation of the pigment particles contained in the dermis tissue such that the fragmented particles can be eliminated by the subject's body. In one particular embodiment, the one or more shock wave pulses have a delivered positive pulse pressure wave of greater than 1.5 MPA and a pulse duration of less than 5 μs. In another particular embodiment, the one or more shock wave pulses have a delivered positive pulse pressure wave of greater than 10 MPA and a pulse duration of less than 1 μs. In other particular embodiments, the one or more shock wave pulses have a delivered negative pulse pressure wave of less than 20 MPA and less than 10 MPA, respectively.

FIG. 3 is a flow chart showing a method of removing permanent tissue markings, such as tattoos and other concretions located within the dermis of a subject, through the use of extracorporeally generated shock waves according to another particular embodiment wherein the focusing location of each of the shock wave pulses is changed so that no consecutive shock wave pulses are focused within the same portion of tissue. The method shown in FIG. 3 is described in connection with the apparatus 2 shown in FIG. 1. Again, this is not meant to be limiting and it is to be understood that the method may be practiced with other suitable apparatus capable of generating and focusing suitable shock waves. Further, as was the case with the method shown in FIG. 2, the method of FIG. 3 is described in connection with the removal of a tattoo. It should be understood, however, that this is not meant to be limiting, and that the method may be employed to remove other concretions located within the dermis of a subject.

The method begins at step 16, wherein an initial target location of the dermis that includes the tattoo is chosen. The applicator 8 is then applied to the surface of the skin at this initial target location. Next, at step 18, a shock wave pulse is generated by the transducer 6. Then, at step 20, the shock wave pulse is focused by the applicator 8 within the dermis tissue at the current dermis target location. At step 22, a determination is made as to whether the treatment is complete. It the answer is yes, the method ends. If, however, the answer is no, meaning that treatment is to continue, then the method proceeds to step 24, wherein a different dermis target location (i.e. different that the immediately previous target location) is selected. Thereafter, the method returns to steps 18 and 20 so that a shock wave pulse will be generated and focused at that target location. Thus, as will be appreciated, the method shown in FIG. 3 will result in no consecutive shock wave pulses being focused within the same portion of tissue.

As noted elsewhere herein, the mechanism of how lithotripsy works in fragmenting kidney stones, which are macroscopic particles, has been discussed in the literature. The use of shock waves for the fragmentation of microscopic particles, including pigment particles which form tattoos, using shock wave pulses has not been described or suggested in the literature. In fact, prior to the present invention, there was no evidence that lithotripsy could be useful to the destruction of microscopic particles. The present invention demonstrates for the first time that microscopic particles such as pigment particles can be made smaller after being treated with shock wave pulses from a device such as apparatus 2.

The inventors believe that the shock wave strikes the pigment particles that make up the tissue marking. At the pigment particle boundary, energy is lost, and this causes small cracks to form on the edge of the pigment particle. The same effect occurs when the shock wave exits the pigment particle. With successive shocks, the cracks open up, and in turn, smaller cracks form within the large cracks. Eventually, the pigment particles are reduced to small particles, which are then absorbed by cells and eliminated.

In removing skin markings according to one or more embodiments of the present invention, the patient would treated with a system, such as apparatus 2, that can deliver extracorporeal shock waves to the tissue site containing the skin markings. Preferably, the system has a transducer and/or coupler that are designed to focus the shock wave in the dermis of the patient, as is the case with the apparatus 2. Preferably, the focus of the shock wave should be greater than 5 mm in diameter and most preferably greater than 10 mm in diameter. The desired shock wave pressure pulse is one that provides strikingly high amplitude combined with short rise time and pulse duration. The positive pulse pressure wave delivered is preferably greater than 1.5 MPA and most preferably greater than 10 MPA with pulse durations of preferably less than 5 μs and most preferably less than 1 μs. In addition, it is thought that the negative pressure wave in lithotripsy is the cause of cavitations resulting in bubbles which is a major source of the pain. Therefore, according to an aspect of an embodiment of the invention, the magnitude of the negative pressure wave is preferably minimized with a delivered negative pressure wave of less than 20 MPA and most preferably less than 10 MPA.

Too cause particle fragmentation, numerous shock wave pulses typically need to be delivered to a specific area. Lithotripsy used for the removal of kidney stones usually requires 100 to 2000 shock wave pulses. For removing pigment particles in the skin, it is believed that an equivalent number of shock wave pulses will be required.

It should be noted that lithotripsy often has pain associated with its use to remove kidney stones. It is believed that this pain is caused by the formation of bubbles following cavitations in the tissue. To minimize the pain associated residual bubbles following cavitation, it is recommended that longer time intervals (>0.5 s) between succeeding pressure pulses be employed. The extended time intervals would provide time for the bubbles after cavitation to become progressively absorbed, therefore minimizing pain.

Moreover, tattoos typically cover large areas of the dermis. Since lithotripsy has a focus area that is relatively small in comparison to the typical tattoo, the lithotripsy will have to be moved to different areas in order to remove the tattoo. As a result, it is recommended that the lithotripsy be focused at different areas of the tattoo between shock wave pulses so that no specific tattoo area is shocked in series. One embodiment of such a method was described in connection with FIG. 3. This has the added benefit of minimizing pain caused by cavitation bubbles by providing time for the bubbles to be reabsorbed in the area of the tissue that was just pulsed. For example, for a tattoo that has an area of 20 mm in diameter and using a system that can focus its shock wave pulse to an area of 10 mm in diameter, the tattoo may be divided into two or three areas or portions to be treated. The system, such as apparatus 2, would provide a shock wave pulse to one area and then be move to a second area, third area, etc. After providing pulses to the different areas, the treatment begins the series again starting with the first area. This process can, according to one particular embodiment, be done automatically through the programming of the system employed, such as by providing the apparatus with a controller that can automatically control the focus area of the shock wave pulses that are generated, such as by automatically controlling and moving the position of the applicator 8. In this way, efficient use of the apparatus 2 is obtained while providing extended interval time between pulses at a specific tattoo area that is being treated. It should be noted that for small tattoos, a single area can be treated. In this case, care must be taken to provide an adequate time interval between pulses so as to minimize the formation of bubbles.

The use of shock wave pulses for the removal of skin markings as described herein has many potential major advantages over the use of lasers. As stated elsewhere herein, in general, laser treatment for tattoo removal is painful. The use of shock wave pulses may provide removal of tattoos with little if any pain. This may be especially true when “wide-focus and low-pressure” extracorporeal shock wave treatment is used. In addition, the use of laser light directed at tissue has been found to cause damage to or destruction of the surrounding tissues. The use of shock waves for tattoo removal should generate little heat and therefore result in little damage or destruction of the surrounding tissues. Finally, laser removal requires multiple treatment sessions (usually five to twenty) with expensive equipment for maximal elimination. Typically, since many wavelengths are needed to treat multicolored tattoos, more than one laser system is needed to remove all the available inks and combination of inks. Even with multiple treatments, laser therapy is usually limited to eliminating only from 50-70% of the tattoo pigment, resulting in a residual smudge. As a result, the overall cost of laser removal is generally prohibitively expensive. The use of shock waves for tattoo removal as described herein on the other hand can be applied to a relatively large area in an efficient manner, therefore reducing the number of treatment sessions that are required. Furthermore, since of shock waves for tattoo removal is not dependent on the absorption of light, separate systems will not be required to remove all the available inks and combination of inks.

The present invention provides a method for the removal of permanent tissue markings, such as tattoos, through the use of extracorporeal shock wave lithotripsy. There may be situations wherein a combination of approaches is used to remove the permanent tissue markings. For example, this invention contemplates the use of method of removal of permanent tissue marking caused by particles through the use of extracorporeal shockwave lithotripsy in combination with laser, wherein the laser is a Q-switch laser.

To demonstrate that lithotripsy can be used to modify pigment particles, an experiment was performed on 5 tattoo inks (black, white, blue, yellow and red) from Inksmith.biz (290-G Applewood Ctr. Pl. #334 Seneca, S.C. 29678) using a Dornier HM-3 ESWL Machine. Approximately 2 ml of each tattoo ink was placed in a rubber bag. Each bag of tattoo ink was then positioned in the blast path of Dornier HM-3 ESWL Machine. Each bag received 1,000 shocks at 20 KV. After the ESWL treatment, both spectrophotometrics analysis and Coulter counter studies were performed on each treated tattoo ink. Tattoo inks of each color that were not shocked were used as controls.

The results of the spectrophotometrics analysis of each ink (1:10,000 dilution) demonstrated that there was a decrease in absorption for yellow, red and blue color tattoo inks after EWSL versus control. Due to wavelength absorption, the black and white colors were not possible to be analyzed with this technique. This change in absorption indicated that the EWSL had effect on the tattoo inks. To demonstrate that ESWL had a direct effect on the particle size of the tattoo ink pigment particles, i.e., caused a decreased mean particle size of each color, Coulter Counter Analysis was performed. The Coulter Counter analysis had a detectable range of 3-3,000 nanoMeter (rim). The treated and control tattoo ink colors were each diluted 1:40,000 prior to analysis. The results of this study are shown in Table 1. The results of the Coulter Counter Analysis indicate that the extracorporeal shock wave lithotripsy caused a decreased mean particle size for each of the tattoo ink colors.

TABLE 1

| Ink | | Mean (nm) | Sd. Dev. (nm) |
|---|---|---|---|
| Black | Control | 116.9 | 42.2 |
| | ESWL | 108.5 | 36.9 |
| White | Control | 380.4 | 162.4 |
| | ESWL | 306.4 | 140.9 |
| Yellow | Control | 394.2 | 162.4 |
| | ESWL | 351.6 | 140.9 |
| Red | Control | 239.2 | 90.7 |
| | ESWL | 225.6 | 83.3 |
| Blue | Control | 150.9 | 64.1 |
| | ESWL | 128.5 | 54.2 |

The present invention thus provides of a method for the removal of permanent tissue markings, such as tattoos and other concretions located within the dermis of a subject, that is markings that is not dependent on the transmission and absorption of laser light. Instead, extracorporeally generated shock waves are employed to efficiently destroy (comminute) the particles which form the concretion, such as the pigment particles which form tissue markings such as tattoos.

While preferred embodiments of the invention have been described and illustrated above, it should be understood that these are exemplary of the invention and are not to be considered as limiting. Additions, deletions, substitutions, and other modifications can be made without departing from the spirit or scope of the present invention. Accordingly, the invention is not to be considered as limited by the foregoing description but is only limited by the scope of the appended claims.

What is claimed is:

1. A method of removing markings from the tissue of a subject, comprising:
generating one or more shock wave pulses external to said subject's body; and
focusing said one or more shock wave pulses within one or more portions of said tissue, each of said one or more portions of said tissue containing at least a portion of said markings.

2. The method according to claim 1, wherein said markings are caused by a plurality of pigment particles contained in said tissue, and wherein said focusing causes fragmentation of the ones of said particles contained in each of said one or more portions of said tissue.

3. The method according to claim 1, wherein each of said one or more shock wave pulses has a delivered positive pulse pressure wave of greater than 1.5 MPA and a pulse duration of less than 5 its.

4. The method according to claim 3, wherein each of said one or more shock wave pulses has a delivered positive pulse pressure wave of greater than 10 MPA and a pulse duration of less than 1 μs.

5. The method according to claim 3, wherein each of said one or more shock wave pulses has a delivered negative pulse pressure wave of less than 20 MPA.

6. The method according to claim 5, wherein each of said one or more shock wave pulses has a delivered negative pulse pressure wave of less than 10 MPA.

7. The method according to claim 1, wherein a location of said focusing of each of said shock wave pulses is changed so that no consecutive shock wave pulses are focused within the same one of said portions of said tissue.

8. The method according to claim 1, wherein said generating is performed using an ultrasonic transducer.

9. The method according to claim 1, wherein said generating is performed using an opto-acoustic transducer.

10. The method according to claim 1, wherein said generating is performed using a spark-gap transducer.

11. The method according to claim 1, wherein each of said shock wave pulses is focused within a diameter greater than 5 mm.

12. The method according to claim 11, wherein each of said shock wave pulses is focused within a diameter greater than 10 mm.

13. The method according to claim 1, further comprising generating laser energy and focusing said laser energy within said one or more portions of said tissue.

14. The method according to claim 13, wherein said laser energy is generated using a Q-switched laser.

15. The method according to claim 1, wherein said one or more shock wave pulses comprise a plurality of shock wave pulses, and wherein said generating comprises spacing the generation of each of said shock wave pulses by at least 0.5 seconds.

16. A method of removing markings from the tissue of a subject, comprising:
selecting an initial target location of said tissue containing a first portion of said markings;
generating a first shock wave pulse external to said subject's body;
focusing said first shock wave pulse within said tissue at said initial target location;
selecting a new target location of said tissue containing a second portion of said markings, said new target location being different from said initial target location;
generating a second shock wave pulse external to said subject's body; and
focusing said second shock wave pulse within said tissue at said new target location.

17. A method of comminuting a concretion located within the dermis of a subject, comprising:
generating one or more shock wave pulses external to said subject's body; and
focusing said one or more shock wave pulses within one or more portions of said dermis where said concretion is located.

18. The method according to claim 17, wherein each of said one or more shock wave pulses has a delivered positive pulse pressure wave of greater than 1.5 MPA and a pulse duration of less than 5 μs.

19. The method according to claim 18, wherein each of said one or more shock wave pulses has a delivered positive pulse pressure wave of greater than 10 MPA and a pulse duration of less than 1 μs.

20. The method according to claim 18, wherein each of said one or more shock wave pulses has a delivered negative pulse pressure wave of less than 20 MPA.

21. The method according to claim 20, wherein each of said one or more shock wave pulses has a delivered negative pulse pressure wave of less than 10 MPA.

22. The method according to claim 17, wherein a location of said focusing of each of said shock wave pulses is changed so that no consecutive shock wave pulses are focused within the same one of said portions of said tissue.

23. The method according to claim 17, wherein said generating is performed using an ultrasonic transducer.

24. The method according to claim 17, wherein said generating is performed using an opto-acoustic transducer.

25. The method according to claim 17, wherein said generating is performed using a spark-gap transducer.

26. The method according to claim 17, wherein each of said shock wave pulses is focused within a diameter greater than 5 mm.

27. The method according to claim 26, wherein each of said shock wave pulses is focused within a diameter greater than 10 mm.

28. The method according to claim 17, further comprising generating laser energy and focusing said laser energy within said one or more portions of said dermis.

29. The method according to claim 28, wherein said laser energy is generated using a Q-switched laser.

30. The method according to claim 17, wherein said one or more shock wave pulses comprise a plurality of shock wave pulses, and wherein said generating comprises spacing the generation of each of said shock wave pulses by at least 0.5 seconds.
